(19) United States
(12) Patent Application Publication
(10) Pub. No.: US 2024/0071632 A1
(43) Pub. Date: Feb. 29, 2024

(54) VIDEO CALL ASSISTANCE FOR EXPERTISE PROFILES

(71) Applicant: Rods&Cones Holding bv, Amsterdam-Duivendrecht (NL)

(72) Inventors: Jan DHEEDENE, Vosselaar (BE); Bruno DHEEDENE, Amsterdam (NL); Benoit QUIRYNEN, Amsterdam-Duivendrecht (NL); Dario VULJANIC, Amsterdam-Duivendrecht (NL); Dalibor KOFJAC, Amsterdam-Duivendrecht (NL)

(21) Appl. No.: 17/893,688

(22) Filed: Aug. 23, 2022

(51) Int. Cl. G16H 80/00

(52) U.S. Cl. CPC G16H 80/00

(57) ABSTRACT

A method for configuring videoconferences for medical procedures, and specifically for an improved invitation management method for remote experts, is disclosed. Remote users can be added to a videoconference by a medical user in a sterile environment, such as an operating room. The medical user can participate in the videoconference. The medical user uses a wearable, hands-free image sensor, such as smart glasses.

### FIELD OF THE INVENTION

The present invention pertains to an improved method for inviting other users, specifically remote experts, based on certain selection criteria, in an efficient manner.

### BACKGROUND

In medical procedures, and other procedures in a sterile environment (such as certain actions in a clean room and similar situations), increasing emphasis is placed on contactless working, and the persons present should avoid interacting with other objects as much as possible, and preferably entirely, particularly with their hands, as they must remain sterile in order to perform certain actions at a later time.

In a medical setting, a surgeon must be sterile before the procedure starts, and then may not interact, or strongly limit their interactions, with anything that is not sterile. However, today's operating rooms contain a large number of medical instruments that can be interacted with, but this jeopardizes sterility.

In addition, medical procedures increasingly involve experts from multiple fields, especially complex procedures. However, it is difficult for all of them to be physically present due to the limited space in an OR, but also because each additional person further jeopardizes sterility, and also makes the preparation for each procedure much more cumbersome and longer.

A solution that is increasingly used is video conferencing, whereby the experts can assist and advise the person (or persons) physically present in the sterile space. In this application, smart glasses or other wearable devices with one or more image sensors are often used, so that the person on site has his hands free, and the remote experts have a similar image to the person on site and can thus better coordinate with each other. It is also advantageous that they can clearly direct the person on site due to the strongly overlapping field of vision between the image sensor (what the remote experts see) and what the person on site sees. However, it remains a problem to configure the videoconference, as this should primarily be done by the person in the sterile environment, who has access to the instrumentation and also has an overview of who is needed during the videoconference.

In many cases, remote experts are used to provide their expertise during a procedure. These experts may not be necessary during the entire procedure, and do not require to scrub in, or be present from the start. Furthermore, during procedures, unexpected circumstances may arise, which require expertise from unexpected sources.

In each of these cases, the medical user in the sterile environment must be able to contact a suitable remote expert to partake in the videoconference, and must be able to do so in an efficient, reliable (objective) fashion, while maintaining sterility of the environment and the medical user.

### SUMMARY OF THE INVENTION

The present invention and embodiments thereof serve to provide a solution to one or more of above-mentioned disadvantages. To this end, the present invention relates to a method for adding remote users to a videoconference by a medical user in a sterile environment, preferably an operating room, said medical user participating in the videoconference, wherein said sterile environment comprises an interaction terminal, said terminal comprising an input means, a display means and a processor, the medical user wearing a wearable, hands-free image sensor.

Preferred embodiments of the device are shown herein.

### DESCRIPTION OF FIGURES

FIG. 1 shows a schematic set-up of a sterile environment with a medical user, instrumentation and remote experts, according to an embodiment according to the present invention.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g., component and do not exclude or preclude the presence of additional, non-recited components, features, element, members, steps, known in the art or disclosed therein.

In what follows, “medical user” or “medical user in loco” or “medical user in the sterile environment” is used to refer to a person with medical qualifications, who is in the sterile environment during the videoconference. Preferably, this medical user is one of the primary participants in the medical procedure at hand, typically the surgeon leading the procedure.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention relates to a method for adding remote users to a videoconference by a medical user in a sterile environment, preferably an operating room, said medical user participating in the videoconference, wherein said sterile environment comprises an interaction terminal, said terminal comprising an input means, a display means and a processor, the medical user wearing a wearable, hands-free image sensor, preferably provided on the user's head, more preferably smart glasses, the method comprising the following steps:
a. initiating a videoconference via a dedicated application or web browser by the medical user or wherein the medical user is invited into the videoconference;
b. accessing a remote expert database, comprising a plurality of remote expert profiles, each associated to a different remote expert, said remote expert profiles comprising at least name information and expertise information for said remote expert, said expertise information comprising a selection of one or more from a list of predefined keywords, said keywords referring to a predefined medical fields, predefined medical procedures, and/or predefined medical devices or systems;
c. inputting a search string via the input means, comprising one or more words and/or abbreviations;
d. matching one or more predefined keywords from the list to the search string;
e. selecting remote experts suitable for the search string from the remote expert database, based on a comparison of the matched keywords for the search string and the expertise information for the remote experts in the remote expert database;
f. generating an optically readable representation, preferably a Quick Response (QR) code, representing the selected remote experts, and displaying said representation on the display means;
g. upon scanning the representation with the wearable, hands-free image sensor, automatically inviting a first remote expert from the selected remote experts into the videoconference.

In the medical field, more and more use is made of remote expertise under the technological developments in wearable image sensor devices, such as smart glasses. These allow remote users to have a similar, if not the same, visual experience as users in loco, without the burden of having to be physically present, which both saves on time for the remote experts as for the hospital itself, and at the same time reducing peripheral work, such as scrubbing in and prepping, while also reducing encumberment due to overly full operating quarters. Finally, by reducing the number of effectively present personnel in the sterile environment, the risk of infections/sterility breach is reduced as well.

However, during such a videoconference between the medical user in loco, and one or more remote experts, there need to be provisions to contact the remote experts easily and efficiently. In some cases, this is predicable, in the sense that one can presume an anesthesist will be necessary for a certain procedure. However, in case of unforeseen circumstances, the medical user needs to be able to shift quickly and contact the right person, without being prepared in advance who to contact.

In doing so, it should be kept in mind that sterility is to be maintained, and that time is of the essence.

As such, the present invention proposes to create a database of remote experts, each with a profile that allows an automated determination of a most suitable candidate for a specific query by the medical user in the sterile environment.

This is accomplished by providing for an interaction terminal with input means, which only requires a limited input from the medical user, namely a search string which identifies their need. This could for instance be “arthroplasty athlete”, indicating the procedure for which assistance is needed is an arthroplasty for an athlete (as opposed to for a senior citizen, which brings forth a different set of parameters to be considered). Often, surgeons specialize in specific target audiences, which can generate particularly useful information from such experts.

Of course, more generic terms can be used, such as “knee”, leading to a much wider range of experts suitable. Most likely, the expert with particular knowledge on arthroplasty for athletes will be suitable as well, and will be present in the larger pool of candidates that are suitable for the above requirement of expertise in “knee” procedures.

The medical user who typically starts the videoconference, or is invited into it, is given a high amount of control and authority in the proceedings, such that they can easily invite other participants, change settings (volume, muting certain participants, control which feeds are displayed), optionally in combination with one or more remote users that participate in the videoconference. The medical user in loco has the best view on what (who) is necessary for further steps, and can make a qualified estimate on what the required expertise and other features are for such remote experts who are to be invited. By using a database of such remote experts, wherein the profiles are clearly defined by expertise, the medical user can quickly find suitable candidates with as little input required from them as possible. The system then proceeds to analyse the search string it received as input, and can automatically match this to predefined keywords (for instance, if the search string reads “medial pterygoid”, a very specific term which may not be present as an established keyword because of its specificity, the system can intelligently match this to fitting, more generic keywords, such as “facial nerve” and/or others under which this could be classified).

The matching can be performed by machine learning models, which improve over time and use, and in particular by human feedback in case of incorrect matching.

After precompiling a list of selected remote experts, the user is then provided with a optically readable representation via the display means, such that the remote expert can be invited upon scanning of the representation by the hands-free image sensor, for instance of the smart glasses. This way, the user can further optimize time expenditure by pre-setting the compiled list and only calling in the remote expert when it is necessary. This allows the list to be precompiled, after which the medical user can scrub in, and only invite their experts when the procedure has started, wholly removing the need to interact with the interaction terminal after scrubbing in. The additionally needed confirmation of scanning the representation furthermore allows for more certainty in that the right contacts are invited, giving the medical user more time to check the basis on which the list of selected remote experts was compiled.

In a preferred embodiment, the method makes use of one or more specific medical nomenclature databases, which comprises a, for instance hierarchical, listing of human body parts down to a very specific level, which can be used to properly match (parts of) the search string to a fitting keyword. The database can furthermore comprise listings of nomenclature on medical profession and/or procedures, to better match the search string to the predefined keywords.

These nomenclature database can also be used to automatically suggest, correct or complete certain predefined keywords based on the start of a word inputted by the medical user (“arthro” can automatically generate one or more options such as “arthroplasty”, “arthroscopy”, “arthrosis”, etc., ranked by ‘popularity’ and/or further context available). The user can then select a desired word, or complete the word further by themselves. This can also be used to correct incorrect spelling of inputted words, via suggestions or via automatic correction.

In a preferred embodiment, the selected remote experts are ranked based on overlap of the matched keywords for the search string with the expertise information for the remote experts.

By having a preset ranking, this simplifies the contact procedure. The first person on the list, i.e., most suitable regarding expertise based on the search string keywords, is contacted. If they do not respond or indicate they are unavailable, the second person on the list is contacted, and so on. The medical user in loco does not need to intervene if the first person is not available, and the methodology immediately invites the second, etc., person on the list, avoiding the necessity for the medical user to intervene once the procedure has commenced.

In a further improved embodiment, the remote expert profiles comprise availability schedules, and the score is furthermore based on said availability schedules, wherein remote experts are not selected from if they are not available at the time of the videoconference.

The remote experts can input, either manually or automatically, an availability schedule for their remote expert profile. This can be done directly, into an app or via a webbrowser through which they can log on into their profile, or indirectly. The latter can for instance be achieved by uploading (manually or automatically) an agenda or time schedule to the database, or even linking such an agenda or time schedule thereto, so the database can update itself automatically.

By doing so, the methodology can improve the ranking of the selected profiles, thereby increasing the relevance of the contacted expert as well as avoiding wasting time by contacting unavailable experts.

The exact implementation in the ranking algorithm can be performed in many variations. For instance, the availability can be an absolute factor, in that a remote expert who is flagged as not available during the videoconference, is wholly removed from the list of selected remote experts. Alternatively, they can be moved to a position after every other available remote experts, irregardless of their expertise ‘score’. In another variation, the availability has a certain weight, which is factored into a total score along with expertise (and potentially other factors, as discussed further on). This way, candidates which are a poor match based on expertise will be ranked lower than unavailable experts with high scores on expertise. This would allow the methodology to contact these unavailable experts with a chance that they in practice can still accept the invitation while officially being set as unavailable. The choice of the weights can be adjusted for each user separately, or even per case, in advance.

In a further preferred embodiment, the remote expert profiles comprise language proficiency information, and the score is furthermore based on a comparison of said language proficiency information and language proficiency information of the medical user, preferably wherein said medical user has an associated remote expert profile in the remote expert database for comparison.

Additional parameters to be taken into account during videoconferences with remote experts, are a shared language in which the expert and medical user in loco is proficient. Again, this can be taken into account in a number of different ways, for instance as an absolute filter (no shared language proficiency=not selected or selected but after each other available selected expert), or in a weighted manner as discussed above. Exact weights for each factor can be set and adapted if necessary. The language proficiency can be added by selection from a preset list (typically a reduced version of all currently spoken languages, for instance a top-10, which can be expanded if necessary, or even added to in case of unrecognized languages). Alternatively, the list can be filled in entirely manually, preferably with autocorrect/autocomplete options. The former holds the preference, as this ensures a correct matching between language proficiencies of the medical user and that of the remote expert.

In a more preferred embodiment, the search string may even comprise language requirements (for instance, if the medical user wants to hold the videoconference in a specific language for teaching purposes and/or for other observers), which are then matched to the language proficiencies of the remote expert profiles, such that the matching thereof is taken into account for the score.

In a further preferred embodiment, the remote expert profiles comprise historical relationship information, wherein the historical relationship information comprises connections between the remote expert profiles regarding preceding collaborative videoconferences, and wherein the score is furthermore based on said historical relationship information.

Historical relationship information describes previous interactions between remote experts. This can show the familiarity with each other, as this usually leads to more efficient future interactions. This can be simply accounted as a tally of interactions, but more details can be included, such as information regarding the interaction (specific procedure, search string used, length of interaction, rating of the interaction, time passed since interaction), which can impact the impact on the score.

A further advantage is that such interactions can be automatically added into the database, as the system has full knowledge over which remote expert is invited into a videoconference with a medical user, and who accepts, and has availability over all of the additional data (search string, length, time, etc.).

The system may also or alternatively allow for manual updates on interactions, which can also allow remote experts to add collaborations that took place outside of the videoconferencing system. This further supports the focus on familiarity between the users as a bonus to the operating efficiency.

In some variations, these previous interactions can be rated, publicly or non-publicly, to indicate whether the interaction was perceived as positive or negative (preferably marked on a scale) by the interacting parties. In the former case, this can increase the score, the latter may decrease the score. The weight of the perceived interaction is (primarily) focused on the perception by the medical user, as they are the searching party for expertise, with the perceived interaction by the remote expert on optionally be taken into account, and preferably with a reduced weight or impact on the score.

In a further preferred embodiment, the remote expert profiles comprise geographical location information, preferably a geographical location schedule, comprising the geographical location of the remote experts, wherein the score is furthermore based on proximity between the geographical location of the remote expert profiles and the geographical location of the medical user, at the time of the videoconference.

Proximity of the remote user to the medical user may be advantageous as well, as it can determine time difference, making sure that the availability does not become an issue over the length of the procedure. Additionally, it can provide some advantages in a match of the mentality or procedural overlap of the remote expert and the medical user in loco. Different countries/states may for instance perform procedures according to a different procedural flow (for legal reasons, cultural reasons, etc.), so some conformity therein would be advantageous.

The above mentioned factors that can be taken into account for the ranking of the selected remote experts can be performed according to a number of algorithms and/or formulas.

For instance, in a first embodiment, the factors are applied sequentially, in that a first ranking is made based on a first score according to one metric (typically expertise or availability), and a second score is made according to a second metric (preferably the other of expertise or availability, or any of the other factors) which handles all tied first scores. After this, a third score can handle further ties, a fourth score, etc. The skilled person will appreciate that different orders are of course possible, for which factor/metric will define each score.

In a second embodiment, a joint score is applied taking into account each factor, with separate weights for each factor. The expertise can for instance account for 50% of the total score as this is crucial, and the availability 25% as this is very important as well, with language proficiency overlap accounting for 15%, historical relationship information for 5% and geographical location information for the remaining 5%. The skilled person will appreciate that different weight distributions can be applied, and that not all factors need necessarily contribute to the total score.

Hybrid systems may exist as well of course with one or more factors, for instance the availability and/or expertise, being applied as an absolute filter (threshold expertise score, for example), with the remaining factors then accounting (and optionally one or more of the absolute filter factors) defining the score for the ranking.

In a preferred embodiment, the method is configured to communicate with an overall planning system, that allows it to automatically collect data on the procedures in which videoconferences are used. For instance, by knowledge of the location/room in which the videoconference is held (that of the medical user in loco), the method may find in the planning or schedule what is scheduled at that time, and can provide this as further input for the selection of the remote experts. For instance, if it recognizes that in the room where the medical user sets up the videoconference, an arthroplasty is scheduled, it can automatically use this as a keyword for the remote experts that are queried, even if the medical user does not provide this context. It can also automatically recognize the presence of other participants in the procedure, and if present in its database, infer knowledge therefrom (for instance, language proficiency, historical relationship information, etc.) to use to select remote experts. Of course, such indirectly collected information and context can be added at a much lower weight than information from the search string that was explicitly provided by the medical user, as it may be too restrictive for the purposes of the medical user (for instance, it may not be important that the procedure is an arthroplasty if an anesthesist is required, while it may be helpful that he or she has some expertise).

In a preferred embodiment, a second remote expert from the selected remote experts is automatically invited into the videoconference if the first remote expert does not accept and/or decline the invitation.

The automatic follow-up to lower ranked remote experts on the list of selected experts, provides the advantages that it doesn't require the medical user in loco to commit further attention to this. The methodology is framed such that it tries to contact the most suitable candidate first (according to a predefined definition of suitability; as mentioned before, numerous options exist with filters, weights, etc.), and then automatically switches to the second most suitable, the third, etc. if the higher ranked remote experts do not accept the invitation. The time during which each remote expert is attempted to be contacted can be set, for instance as 1 minute, 2 minutes, 5 minutes or longer, depending on the urgency.

In some variations, multiple selected remote experts can be attempted to be contacted essentially simultaneously, especially in cases where a high amount of comparably scored remote experts are selected. For instance, each remote expert within a certain range of a predefined score or with a certain minimal score may be considered as suitable, and can be contacted at once, with the first respondent entering the videoconference. For instance, the medical user (or another user with control over the videoconference system) can set the system as accepting any remote expert with a score over 75, which can mean that all remote experts with scores over 75 can be contacted at once. If none respond, then a lower bracket can be contacted (in group or one by one).

In some occasions, even multiple respondent remote experts can be allowed into the videoconference, depending on the settings that can be configured by the medical user.

In a preferred embodiment, the inputted search string is autocorrected and/or autocompleted before the step of matching keywords to the inputted search string. By using autocorrect, it is ensured that the medical user has awareness over the ‘conversion’ of the search string that is effectively considered in the matching algorithm, so he or she may make adjustments if desired.

For autocomplete, similar arguments apply, besides the improved ease for the medical user, reducing the amount of actual interactions necessary with the input means to input their search string, while ensuring that their input will be actually recognized.

In a preferred embodiment, the remote experts are selected based on whether or not they are online.

As mentioned before, availability is a key feature that can dictate the ranking of the remote experts that are selected. The methodology can be supported by an app or program, where the remote experts can log on, giving the system means to actually determine in real-time whether or not the remote experts are available. In a first version, it can base this simply on their status as online or not online, but of course more particular statuses can be implemented, such as ‘busy’, ‘temporarily absent’, ‘do not disturb’, etc., which allow for more complex settings. A remote expert who is ‘busy’ may still be contacted, but will be ranked lower than an ‘available’ remote expert, but higher than a remote expert who is offline, or set as ‘do not disturb’. Weights and/or filters can be applied based on their actual real-time status.

All of the above can further finetune the efficiency of contacting the right people, who can actually participate in the videoconference.

In a preferred embodiment, the expertise information comprises a selection of at least one or more from a sublist of occupational keywords, indicating the medical position of the remote expert, such as surgeon, anesthetist, nurse and/or others.

This adds a further dimension to the functionalities for the medical user to specify who they need as a remote expert.

In a preferred embodiment, the search string can be input by selection from the list of predefined keywords, wherein said predefined keywords are displayed on the display means, preferably wherein the predefined keywords are grouped in groups of related predefined keywords, said groups preferably comprising at least predefined medical field, predefined medical procedure, and predefined medical devices, and more preferably comprising subgroups of related predefined keywords in at least one or more of said groups.

The medical user can select predefined keywords by interaction with the groups, and optionally with the subgroups, and subsequently the predefined keywords to be selected.

By grouping the keywords, the medical user can have a better overview on what their options are, to avoid giving a too specific term or a too general term, resulting in an ineffective selection of remote experts. This can result in more specific and thus more suitable candidates being selected. At each point, the medical user can decide to choose a full group, subgroup, or a specific keyword in a subgroup (or even down to deeper levels of subsubgroups, etc.).

This furthermore can reduce the number of physical interactions, as for instance typing out the full word is no longer required, especially useful since medical terms are often very long.

It can for instance also allow the medical user to be shown a preliminary number of selected remote experts that would fall under the search string at each moment, allowing the medical user to specify further if they have a large amount of hits, or to quit at the right moment.

In a further preferred embodiment, the interaction with the groups and the predefined keywords, and optionally the subgroups, is effected via gesture recognition on the medical user by a second image sensor in the sterile environment.

Gesture recognition would wholly take away the need for physical interaction with the input means, which could break the sterility, requiring rescrubbing. Voice command input is likewise an option that can be implemented separately or in combination with one or more other input types, such as touchscreen, keyboard, gesture recognition, eye tracking, etc.

In an even further preferred embodiment, the second image sensor is the wearable, handsfree image sensor.

In a preferred embodiment, the remote experts are enabled to modify the expertise information associated to their remote expert profile, and preferably modify one or more of the availability schedules, language proficiency information, historical relationship information and/or geographical location information associated to their remote expert profile.

In some variations, authorized supervising users can be required to confirm some or all of these changes. For instance, the historical relationship information can be edited by the remote experts, as this can allow them to rate collaborations. Language proficiencies can either be set manually, but could likewise also require provision of a certificate or similar documents, which can then be inputted in the system by an authorized supervisor. Some things, such as availability schedules are obviously configurable by the remote experts without confirmation necessary.

In a further preferred embodiment, the selected remote experts are ranked primarily based on the overlap of the matched keywords for the search string with the expertise information for the remote experts, and secondarily in case of tie based on the availability schedules, language proficiency information, historical relationship information and/or geographical location information of the selected remote experts.

In an even further preferred embodiment, the selected remote experts are ranked primarily based on the overlap of the matched keywords for the search string with the expertise information for the remote experts, and secondarily in case of tie based on the availability schedules, and tertiarily based on language proficiency information, historical relationship information and/or geographical location information of the selected remote experts.

In an alternative embodiment, the selected remote experts are ranked primarily based on the availability schedules, and secondarily based on the overlap of the matched keywords for the search string with the expertise information for the remote experts, and tertiarily based on one or more of language proficiency information, historical relationship information and/or geographical location information of the selected remote experts. As mentioned previously, expertise and availability are crucial parameters for finding the right remote expert.

The invention is further described by the following non-limiting examples which further illustrate the invention, and are not intended to, nor should they be interpreted to, limit the scope of the invention.

The present invention will be now described in more details, referring to examples that are not limitative.

### EXAMPLES

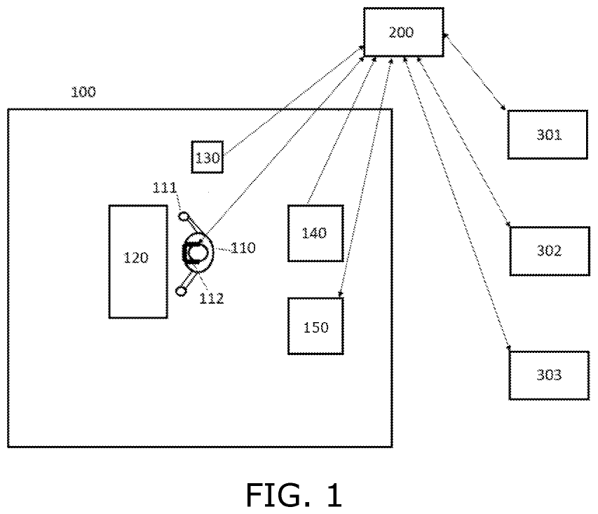

FIG. 1 shows a schematic configuration of actors in a videoconference. In and around the sterile environment (100), in this case operating quarters, an operating table (120) is centrally positioned. A medical user (110), for instance a surgeon, is positioned centrally at said table, and is wearing smartglasses (112), which comprise a wearable, hands-free image sensor. The smartglasses (112) send and receive data in the videoconference. The sterile environment (100) furthermore may comprise one or more medical instruments (140), which in this case participate in the videoconference and share a certain data-output (video, audio and/or others) in said videoconference, and can optionally also receive data via said link to the videoconference. Furthermore, a ‘second’ wearable, hands-free image sensor (130) may or may not be present in the sterile environment, which can be used to identify gestures from the user, for instance. The sterile environment (100) furthermore comprises an interaction means (150), via which a person in the sterile environment (100), typically the medical user (110), can provide input for the search string.

The devices participating in the videoconference communicate with/via an intermediary server (200), through which remote experts (301, 302, 303) can be communicated with. In cases where the videoconference only hosts one additional remote expert, direct communication between the remote expert and the medical user is preferred, skipping the intermediary server.

In the present invention, the inputted search string is compared and matched versus the database of remote expert profiles, to generate a selection of suitable remote experts. As mentioned, a number of further factors, aside from the search string, can come into play for this selection. The selection is preferably ranked according to level of suitability.

In a first action, the first ranked remote expert (301) is contacted and invited into the videoconference. If he or she declines or does not respond within a certain timeframe, the invitation is dropped, and the second ranked remote expert (302) is contacted and invited. If necessary, a third (303) and even further ranked remote experts can be contacted.

In a variation, the N first ranked selected remote experts, or all selected remote experts with a score exceeding a threshold, can be contacted at once.

The present invention should not be construed as being limited to the embodiments described above and certain modifications or changes may be added to the examples described without having to re-evaluate the appended claims. For example, the present invention has been described with reference to medical procedures, but it should be understood that the invention can be applied to, for example, hazardous operations, such as explosives dismantling or maintenance in high radioactivity environments, maintenance of deep-sea systems, hard-to-access systems or equipment, and others. In any of these situations, the expertise of several people would be extremely beneficial, but it is either impossible or too high an additional risk.

1. A method for adding remote users to a videoconference by a medical user in a sterile environment, the medical user participating in the videoconference, wherein the sterile environment comprises an interaction terminal, the terminal comprising an input means, a display means and a processor, the medical user wearing a wearable, hands-free image sensor, the method comprising the following steps:
a. initiating a videoconference via a dedicated application or web browser by the medical user or wherein the medical user is invited into the videoconference;
b. accessing a remote expert database, comprising a plurality of remote expert profiles, each associated to a different remote expert, the remote expert profiles comprising at least name information and expertise information for the remote expert, the expertise information comprising a selection of one or more from a list of predefined keywords, the keywords referring to a predefined medical fields, predefined medical procedures, and/or predefined medical devices or systems;
c. inputting a search string via the input means, comprising one or more words and/or abbreviations;
d. matching one or more predefined keywords from the list to the search string;
e. selecting remote experts suitable for the search string from the remote expert database, based on a comparison of the matched keywords for the search string and the expertise information for the remote experts in the remote expert database;
f. generating an optically readable representation representing the selected remote experts, and displaying the representation on the display means; and
g. upon scanning the representation with the wearable, hands-free image sensor, automatically inviting a first remote expert from the selected remote experts into the videoconference.

2. The method according to claim 1, wherein the selected remote experts are ranked based on overlap of the matched keywords for the search string with the expertise information for the remote experts.

3. The method according to claim 2, wherein the remote expert profiles comprise availability schedules, and the score is further based on the availability schedules, wherein remote experts are not selected from if they are not available at the time of the videoconference.

4. The method according to claim 2, wherein the remote expert profiles comprise language proficiency information, and the score is furthermore based on a comparison of the language proficiency information and language proficiency information of the medical user.

5. The method according to claim 2, wherein the remote expert profiles comprise historical relationship information, wherein the historical relationship information comprises connections between the remote expert profiles regarding preceding collaborative videoconferences, and wherein the score is further based on the historical relationship information.

6. The method according to claim 2, wherein the remote expert profiles comprise geographical location information comprising the geographical location of the remote experts, wherein the score is further based on proximity between the geographical location of the remote expert profiles and the geographical location of the medical user, at the time of the videoconference.

7. The method according to claim 1, wherein a second remote expert from the selected remote experts is automatically invited into the videoconference if the first remote expert does not accept and/or decline the invitation.

8. The method according to claim 1, wherein the inputted search string is autocorrected and/or autocompleted before the step of matching keywords to the inputted search string.

9. The method according to claim 1, wherein the remote experts are selected based on whether or not they are online.

10. The method according to claim 1, wherein the expertise information comprises a selection of at least one or more from a sublist of occupational keywords, indicating the medical position of the remote expert.

11. The method according to claim 1, wherein the search string is inputted by selection from the list of predefined keywords, wherein the predefined keywords are displayed on the display means, wherein the predefined keywords are grouped in groups of related predefined keywords, the groups comprising at least a predefined medical field, predefined medical procedure, and predefined medical devices, and further comprising subgroups of related predefined keywords in at least one or more of the groups,
wherein the medical user selects predefined keywords by interaction with the groups, and optionally with the subgroups, and subsequently the predefined keywords to be selected.

12. The method according to claim 11, wherein the interaction with the groups and the predefined keywords, and optionally the subgroups, is effected via gesture recognition on the medical user by a second image sensor in the sterile environment.

13. The method according to claim 12, wherein the second image sensor is the wearable, handsfree image sensor.

14. The method according to claim 1, wherein the remote experts are enabled to modify the expertise information associated to their remote expert profile, and modify the availability schedules, language proficiency information, historical relationship information and/or geographical location information associated to their remote expert profile.

15. The method according to claim 2, wherein the selected remote experts are ranked primarily based on the overlap of the matched keywords for the search string with the expertise information for the remote experts, and secondarily in case of tie based on the availability schedules, language proficiency information, historical relationship information and/or geographical location information of the selected remote experts.

16. The method according to claim 1, wherein the hands-free image sensor is smart glasses.

17. The method according to claim 1, wherein the optically readable representation is a Quick Response (QR) code.

18. The method according to claim 4, wherein the medical user has an associated remote expert profile in the remote expert database for comparison.
